(19) United States
(12) Patent Application Publication
(10) Pub. No.: US 2008/0171395 A1
(43) Pub. Date: Jul. 17, 2008

(54) Column packing and concentrating column for the analysis of metallic elements

(75) Inventors: Xinshen Zhang, Chengdu (CN); Xiaoping Jiang, Chengdu (CN)

Correspondence Address: CONNOLLY BOVE LODGE & HUTZ, LLP, P O BOX 2207, WILMINGTON, DE, 19899, US

(73) Assignee: Sichuan University, Chengdu (CN)

(21) Appl. No.: 11/824,978

(22) Filed: Jul. 3, 2007

(30) Foreign Application Priority Data
Jan. 15, 2007 (CN) 200710048265.9

(51) Int. Cl. G01N 33/20; B01D 35/30

(52) U.S. Cl. 436/73; 422/243

(57) ABSTRACT

The present invention relates to a column packing and concentrating columns for the analysis of metallic elements, wherein the column packing is formed from swollen and cleaned macroporous adsorbent resins of high, medium or non-polarity via dynamic on-column treatment or static immersion treatment using treating solution I or treating solution II; said treating solution I is a mixed aqueous solution of 0.02˜0.06 g/L of 2-(5-bromo-2-pyridyl azo)-5-diethylamino phenol, 1.60˜1.90 g/L of Na₂B₄O₇.10H₂O, 6.0×10⁻³˜8.0×10⁻³ mol/L of HCl, and polyethylene glycol octyl phenyl ether with a volume percent of 0.3˜0.8%; said treating solution II is a mixed aqueous solution of 0.04˜0.10 g/L of 2-(5-bromo-2-pyridyl azo)-5-diethylamino phenol, 0.05˜0.08 mol/L of glycin, 0.05˜0.08 mol/L of NaOH, 0.010˜0.015 mol/L of NaCl, and polyethylene glycol octyl phenyl ether with a volume percent of 0.3˜0.8%.

### FIELD OF THE INVENTION

The present invention relates to column packings and concentrating columns for the analysis of metallic elements, in particular relates to column packings and concentrating columns for the analysis of metal element contained in seawater, estuarine water and river water.

### BACKGROUND OF THE INVENTION

Column packing is an important component of a concentrating column, which is a key part of instruments for analysis of metallic elements contained in seawater, estuarine water and river water. For example, concentrating columns can be coupled with other instruments such as ion chromatography analyzer, flow injection analyzer, inductively coupled plasma emission spectra—mass spectra associated analyzer, atomic absorption spectrophotometer, so as to enrich and analyze metallic elements in samples. In concentrating columns, column packings are used for the purposes of enrichment and separation. Therefore, researches concerning column packings and concentrating columns are attracting continuous attention.

China patent ZL 200410040361.5 discloses a concentrating column for the trace analysis of metallic elements, wherein the column packing of the concentrating column is formed from swollen and cleaned macroporous adsorbent resins of high, medium or non-polarity, which were subjected to dynamic on-column treatment or static immersion treatment using a mixed solution of 3 mol/L NH₄OH −1 mol/L HAC—3.0˜6.0×10⁻⁴ mol/L pyridine-(2-azo-4-)resorcin or a mixed solution of 0.5 mol/L Na₂HPO₄—1.0˜2.0×10⁻⁴ mol/L pyridine-(2-azo-4-)resorcin. This column packing and concentrating column can simplify the treatment of samples and the configuration of the devices for treating the samples, so as to enhance the processing speed, and adsorb Hg²⁺ on the concentrating column. However, during the on-column treatment or immersing treatment of the polar or medium-polar or non-polar macroporous resins, large amounts of treating agents are required (mixed solution of 3 mol/L NH₄OH—1 mol/L HAC-3.0˜6.0×10⁻⁴ mol/L pyridine-(2-azo-4-) resorcin or mixed solution of 0.5 mol/L Na₂HPO₄—1.0˜2.0×10⁻⁴ mol/L pyridine-(2-azo-4-)resorcin), which interfere the wide application of column packings and further reduction of the cost of concentrating columns.

### SUMMARY OF THE INVENTION

The purpose of the present invention is to provide a novel column packing and concentrating columns using said column packing, so as to overcome the shortages of prior art. While maintaining the sample treatment process and simplifying the structures of sample treatment devices, as well as having the advantage of absorbing various metallic elements, the present invention further decreases the cost of column packing, extends the application of column packings and concentrating columns.

The technical solution of the present invention comprises: subjecting swollen and cleaned macroporous adsorbent resins of high, medium or non-polarity to dynamic on-column treatment or static immersion treatment, wherein treating solution I or treating solution II is used.

The column packing useful for the analysis of metallic elements according to the present invention is formed from the swollen and cleaned macroporous adsorbent resins of high, medium or non-polarity by immersing treatment using treating solution I or treating solution II.

The concentrating column according to the present invention consists essentially of a column body, filtration membranes installed at the inner sides of the inlet and outlet of the column body, and a column packing packed inside the column body, wherein the column packing is formed from swollen and cleaned macroporous adsorbent resins of high, medium or non-polarity via dynamic on-column treatment or static immersion treatment using treating solution I or treating solution II.

The swelling of said polar or medium-polar or non-polar macroporous adsorptive resins is achieved by immersing them in ethanol at room temperature for at least 8 hours. The resins are then rinsed with deionized water.

Treating solution I is a mixed aqueous solution of 0.02-0.06 g/L of 2-(5-bromo-2-pyridyl azo)-5-diethylamino phenol, 1.60-1.90 g/L of Na₂B₄O₇.10H₂O, 6.0×10⁻³-8.0×10⁻³ mol/L of HCl, and polyethylene glycol octyl phenyl ether (OP emulsifying agent) with a volume percent of 0.3-0.8%. Treating solution II is a mixed aqueous solution of 0.04-0.10 g/L of 2-(5-bromo-2-pyridyl azo)-5-diethylamino phenol, 0.05-0.08 mol/L of glycine, 0.05-0.08 mol/L of NaOH, 0.010-0.015 mol/L of NaCl, and polyethylene glycol octyl phenyl ether (OP emulsifying agent) with a volume percent of 0.3-0.8%. The chemicals used in the preparation of treating solution I and treating solution II are all conventional chemicals, which are commercially available.

The static immersion treatment of said swollen and cleaned macroporous adsorbent resins of high, medium or non-polarity comprises placing the swollen and cleaned macroporous adsorbent resins of high, medium or non-polarity into treating solution I or treating solution II, immersing at room temperature for at least 4 minutes, and then rinsing it with deionized water until the rinsing water becomes colorless. During the static immersion treatment, the volume ratio between the swollen and cleaned macroporous adsorbent resins of high, medium or non-polarity and the treating solution I or treating solution II is 1:2˜1:4.

The dynamic on-column treatment of said swollen and cleaned macroporous adsorbent resins of high, medium or non-polarity comprises packing the swollen and cleaned macroporous adsorbent resins of high, medium or non-polarity into the column, then pumping treating solution I or treating solution II into the concentrating column directly or after 2˜4 folds dilution using deionized water, until the effluent from the concentrating column changes from colorless to light red, then pumping deionized water into the concentrating column, until the effluent from the concentrating column changes from light red to colorless.

The present invention provides the following advantages.

1. To complete the static immersion treatment or dynamic on-column treatment, the cleaned, polar or medium-polar or non-polar macroporous adsorptive resins of the present invention need less time (which is between 1/20 and 1/10 of the time needed in China Patent ZL 200410040361.5, see Example 7) and consume very small amount of treating solutions (which is between 1/20 and 1/10 of that of China Patent ZL 200410040361.5, see Example 7). As a result, the cost of the column packing is reduced.

2. Benefited from the reduction of the column packing cost, the column packing can be used for treating environmental water, such as removing the harmful metallic elements including Cd, Hg and the like.

3. Various metallic elements, including Cu, Zn, Cd, Hg, Fe, Mn, Co, Ni and the like, can be adsorbed by the column packing.

4. By using the concentrating column with the column packing according to the present invention, the test samples are not required to experience complexing treatment. As a result, the procedures for treating the samples and the structure of the sample processing device are simplified.

5. By using the concentrating column with the column packing according to the present invention, a solution of 5-8×10⁻³ mol/L of HNO₃ or a mixed solution of 0.010 mol/L of oxalic acid-0.0075 mol/L of citric acid-1.15 g/L LiOH can be used as the desorbing solution.

6. The concentrating column according to the present invention can be combined with instruments like ion chromatography analyzer, flow injection analyzer, inductively coupled plasma emission spectra-mass spectra coordinating analyzer, atomic absorption spectrophotometer and the like.

### DESCRIPTION OF THE DRAWINGS

FIG. 1 is a structural diagram of the concentrating column for analyzing trace metal elements according to the present invention.

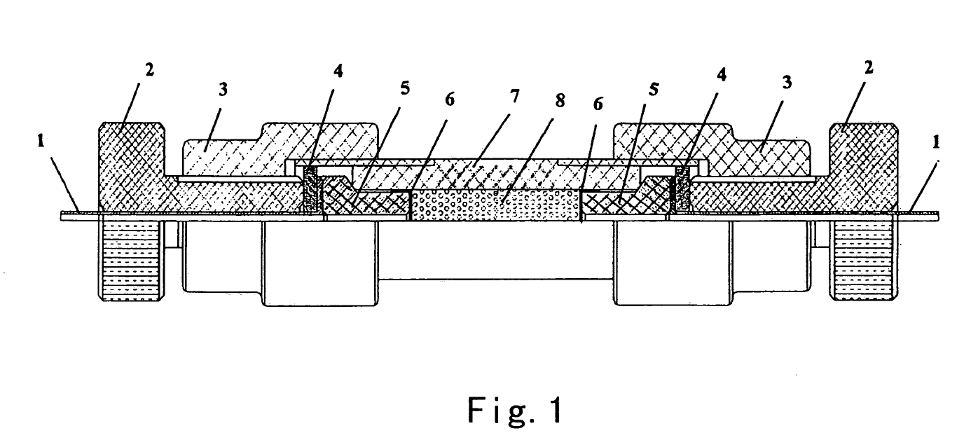

In FIG. 1, the numerals represent: 1—conduit, 2—conduit-holding bolt, 3—connection cannula, 4—sealing ring, 5—plug, 6—filtration membrane, 7—column tube, 8—column packing.

### DETAILED DESCRIPTION OF THE INVENTION

It is to be noted that the term “a” or “an” entity refers to one or more of that entity.

The following examples are provided for illustrative purposes only and are not intended to limit the scope of the invention.

### Example 1

The column packing of this example was formed from non-polar macroporous adsorptive resins through static immersion treatment. The non-polar macroporous adsorptive resins were selected from Amberlite XAD-1˜Amberlite XAD-5 (obtained from Rohm&Hass company, US) and D3520 (obtained from Nankai University, Tianjin, China). The processing steps were as follows:

1. Immersion, Swelling and Cleansing Treatments

The foregoing non-polar macroporous adsorptive resins were immersed into ethanol in a container at 25° C. (the amount of ethanol is not strictly limited, so long as it flooded the non-polar macroporous adsorptive resins completely). An immersion time of 8 hours was enough for swelling. The swelled resins were then rinsed with deionized water, until there was no ethanol in the resins.

2. Static Immersion Treatment with Treating Solution I

Treating solutions were prepared according to the formulations shown in Table 1, using 2-(5-bromo-2-pyridineazo)-5-diethylamino phenol, Na₂B₄O₇.10H₂O, HCl, polyethylene glycol octyl phenyl ether (OP emulsifying agent) and deionized water as raw materials. And then immersed, swelled and purged non-polar macroporous adsorptive resins were respectively placed into containers containing the foregoing treating solutions, wherein the volume ratio between the non-polar macroporous adsorptive resins and the treating solution in each container is 1:4. The temperatures and immersion times were shown in Table 1. After immersion treatment, the resins were rinsed with deionized water until the rinsing water became colorless, so that the column packing according to the present invention was obtained.

TABLE 1

| | Immersion | Immersion |
|---|---|---|
| | Temperature | Time |
| Treating Solutions | (° C.) | (min) |
| 0.02 g/L of 2-(5-bromo-2-pyridineazo)-5- | 25 | 5 |
| diethylamino phenol, 1.90 g/L of | | |
| Na₂B₄O₇•10H₂O, 6.0 × 10⁻³ mol/L HCl, | | |
| 0.8% polyethylene glycol octyl phenyl ether | | |
| (OP emulsifying agent). | | |
| 0.03 g/L of 2-(5-bromo-2-pyridineazo)-5- | 25 | 4 |
| diethylamino phenol, 1.80 g/L of | | |
| Na₂B₄O₇•10H₂O, 6.5 × 10⁻³ mol/L | | |
| HCl, 0.6% of polyethylene glycol octyl phenyl | | |
| ether (OP emulsifying agent). | | |
| 0.04 g/L of 2-(5-bromo-2-pyridineazo)-5- | 25 | 4 |
| diethylamino phenol, 1.70 g/L | | |
| Na₂B₄O₇•10H₂O, 7.0 × 10⁻³ mol/L HCl, | | |
| 0.4% polyethylene glycol octyl phenyl ether | | |
| (OP emulsifying agent). | | |
| 0.06 g/L 2-(5-bromo-2-pyridineazo)-5- | 25 | 4 |
| diethylamino phenol, 1.60 g/L | | |
| Na₂B₄O₇•10H₂O, 8.0 × 10⁻³ mol/L HCl, | | |
| 0.3% polyethylene glycol octyl phenyl ether | | |
| (OP emulsifying agent). | | |

3. Static Immersion Treatment with Treating Solution Ii

Treating solutions were prepared according to the formulations shown in Table 2, using 2-(5-bromo-2-pyridyl azo)-5-diethylamino phenol, glycin, NaOH, NaCl, polyethylene glycol octyl phenyl ether (OP emulsifying agent) and deionized water as raw materials. And then immersed, swelled and purged non-polar macroporous adsorptive resins were respectively placed into containers containing the foregoing treating solutions, wherein the volume ratio between the non-polar macroporous adsorptive resins and the treating solution in each container is 1:2. The temperatures and immersion times were shown in Table 2. After immersion treatment, the resins were rinsed with deionized water until the rinsing water became colorless, and as a result column packings according to the present invention were obtained.

TABLE 2

| | Immersion | Immersion |
|---|---|---|
| | Temperature | Time |
| Treating Solutions | (° C.) | (min) |
| 0.04 g/L 2-(5-bromo-2-pyridineazo)-5-diethylamino phenol, | 25 | 5 |
| 0.08 mol/L glycine, 0.08 mol/L NaOH, 0.015 mol/L NaCl, | | |
| 0.8% polyethylene glycol octyl phenyl ether (OP emulsifying | | |
| agent). | | |
| 0.06 g/L 2-(5-bromo-2-pyridineazo)-5-diethylamino phenol, | 25 | 5 |
| 0.06 mol/L glycine, 0.07 mol/L NaOH, 0.012 mol/L NaCl, | | |
| 0.6% polyethylene glycol octyl phenyl ether (OP emulsifying | | |
| agent). | | |
| 0.08 g/L 2-(5-bromo-2-pyridineazo)-5-diethylamino phenol, | 25 | 4 |
| 0.05 mol/L glycine, 0.06 mol/L NaOH, 0.012 mol/L NaCl, 0.5% | | |
| polyethylene glycol octyl phenyl ether (OP emulsifying agent). | | |
| 010 g/L 2-(5-bromo-2-pyridineazo)-5-diethylamino phenol, | 25 | 4 |
| 0.05 mol/L glycine, 0.05 mol/L NaOH, 0.010 mol/L NaCl, | | |
| 0.3% polyethylene glycol octyl phenyl ether (OP emulsifying | | |
| agent). | | |

### Example 2

The concentrating column of Example 2 has a structure as shown in FIG. 1, it consists mainly of column body, filtration membranes (6) and column packing (8). The column body is made of nylon and comprises column tube (7), plugs (5) at both ends of the inside of column tube, conduit-holding bolts (2) putting pressure on the plugs, and connection cannulas (3) connecting column tube and conduit-holding bolts. Conduits (1) are fixed in the central holes of conduit-holding bolts (2). The insertion ends of conduits (1) have discs in close contact with the plugs. The sealing rings (4) lie between the discs and the end surface of conduit-holding bolts. Conduits (1) are connected to the central holes of plugs (5) to form a channel for fluid input and output. Filtration membranes (6) are made of nylon taffeta and fixed respectively at the end surface of the insertion sections of the plugs at both ends of the inside of column tube. Column packing (8), which is prepared with non-polar macroporous adsorbent resin Amberlite XAD-1˜Amberlite XAD-5 (obtained from Rohm&Hass company, US) and D3520 (obtained from Nankai University, Tianjin, China) through dynamic on-column treatment or static immersion treatment using treating solution I or treating solution II, are packed into the inside of column tube. Size of the concentrating column is φ5×30 mm.

1. Dynamic On-Column Treatment with Treating Solution I

(1) Preparation of the Mixed Solution

Treating solutions were prepared according to the formulations shown in Table 3, using 2-(5-bromo-2-pyridineazo)-5-diethylamino phenol, Na₂B₄O₇.10H₂O, HCl, polyethylene glycol octyl phenyl ether (OP emulsifying agent) and ionized water as raw materials.

(2) Packing of the Concentrating Column

After immersion, swelling and cleansing treatments, the non-polar macroporous adsorbent resin was added into the column body via a dropper, and washed with deionized water. The swelling and cleaning of the non-polar macroporous adsorptive resins were conducted in a similar manner as in Example 1.

(3) On-Column Treatment

The treating solutions undiluted or diluted with deionized water according to Table 3 were pumped into the concentrating column at flow rates listed in Table 3 at room temperature (25° C.) until the effluent from concentrating column changed from colorless to light red. And then deionized water was pumped through to wash the concentrating column until the effluent turned from light red to colorless, as a result concentrating columns according to the present invention were obtained.

TABLE 3

| Size of the | | | Flow rate on | |
|---|---|---|---|---|
| concentrating | | Without dilution or | column | Time on column |
| column | Treating Solutions | folds of dilution | (ml/min) | (min) |
| φ5 × 30 mm | 0.02 g/L 2-(5-bromo-2-pyridineazo)-5-diethylamino | Without dilution | 1.0 | 2 |
| | phenol, 1.90 g/L Na₂B₄O₇•10H₂O, 6.0 × 10⁻³ mol/L HCl, | | | |
| | 0.8% polyethylene glycol octyl phenyl ether (OP | | | |
| | emulsifying agent). | | | |
| φ5 × 30 mm | 0.03 g/L 2-(5-bromo-2-pyridineazo)-5-diethylamino | 2 folds | 1.0 | 3 |
| | phenol, 1.80 g/L Na₂B₄O₇•10H₂O, 6.5 × 10⁻³ mol/L HCl, | | | |
| | 0.6% polyethylene glycol octyl phenyl ether (OP | | | |
| | emulsifying agent). | | | |
| φ5 × 30 mm | 0.04 g/L 2-(5-bromo-2-pyridineazo)-5-diethylamino | 3 folds | 1.0 | 3 |
| | phenol, 1.70 g/L Na₂B₄O₇•10H₂O, 7.0 × 10⁻³ mol/L HCl, | | | |
| | 0.4% polyethylene glycol octyl phenyl ether (OP | | | |
| | emulsifying agent). | | | |
| φ5 × 30 mm | 0.06 g/L 2-(5-bromo-2-pyridineazo)-5-diethylamino | 4 folds | 1.0 | 3 |
| | phenol, 1.60 g/L Na₂B₄O₇•10H₂O, 8.0 × 10⁻³ mol/L HCl, | | | |
| | 0.3% polyethylene glycol octyl phenyl ether (OP | | | |
| | emulsifying agent). | | | |

Note:
The “time on column” in Table 3 refers to the time period during which a treating solution was pumped into the concentrating column.

2. Dynamic On-Column Treatment with Treating Solution Ii

(1) Preparation of the Mixed Solution

Treating solutions were prepared according to the formulations shown in Table 4, using 2-(5-bromo-2-pyridineazo)-5-diethylamino phenol, glycine, NaOH, NaCl, polyethylene glycol octyl phenyl ether (OP emulsifying agent) and ionized water as raw materials.

(2) Packing of the Concentrating Column

After immersion, swelling and cleansing treatments, the non-polar macroporous adsorbent resins were added into the column body via a dropper, and washed with deionized water. The swelling and cleaning of the non-polar macroporous adsorptive resins were conducted in a similar manner as in Example 1.

(3) On-Column Treatment

The treating solutions undiluted or diluted with deionized water according to Table 4 were pumped into the concentrating column at flow rates listed in Table 4 at room temperature (25° C.) until the effluent from concentrating column changed from colorless to light red. And then deionized water was pumped through to wash the concentrating column until the effluent turned from light red to colorless, as a result concentrating columns according to the present invention were obtained.

TABLE 4

| Size of the | | | Flow rate on | Time on |
|---|---|---|---|---|
| concentrating | | Without dilution or | column | column |
| column | Treating Solutions | folds of dilution | (ml/min) | (min) |
| φ5 × 30 mm | 0.04 g/L 2-(5-bromo-2-pyridineazo)-5-diethylamino | Without dilution | 1.0 | 2 |
| | phenol, 0.08 mol/L glycine, 0.08 mol/L NaOH, 0.015 mol/L | | | |
| | NaCl, 0.8% polyethylene glycol octyl phenyl ether (OP | | | |
| | emulsifying agent). | | | |
| φ5 × 30 mm | 0.06 g/L 2-(5-bromo-2-pyridineazo)-5-diethylamino | Without dilution | 1.0 | 2 |
| | phenol, 0.06 mol/L glycine, 0.07 mol/L NaOH, 0.012 mol/L | | | |
| | NaCl, 0.6% polyethylene glycol octyl phenyl ether (OP | | | |
| | emulsifying agent). | | | |
| φ5 × 30 mm | 0.08 g/L 2-(5-bromo-2-pyridineazo)-5-diethylamino | 2 folds | 1.0 | 2 |
| | phenol, 0.05 mol/L glycine, 0.06 mol/L NaOH, 0.012 mol/L | | | |
| | NaCl, 0.5% polyethylene glycol octyl phenyl ether (OP | | | |
| | emulsifying agent). | | | |
| φ5 × 30 mm | 0.10 g/L 2-(5-bromo-2-pyridineazo)-5-diethylamino | 2 folds | 1.0 | 2 |
| | phenol, 0.05 mol/L glycine, 0.05 mol/L NaOH, 0.010 mol/L | | | |
| | NaCl, 0.3% polyethylene glycol octyl phenyl ether (OP | | | |
| | emulsifying agent). | | | |

Note:
The “time on column” in Table 4 refers to the time period during which a treating solution was pumped into the concentrating column.

3. Static Immersion Treatment with Treating Solution I

After immersion, swelling and cleansing treatments, the non-polar macroporous adsorbent resins were subjected to static immersion treatment with treating solution I in a similar manner as in Example 1. The column packing thus obtained was added into the column body via a dropper, and thus a concentrating column was obtained.

4. Static Immersion Treatment with Treating Solution II

After immersion, swelling and cleansing treatments, the non-polar macroporous adsorbent resins were subjected to static immersion treatment with treating solution II in a similar manner as in Example 1. The column packing thus obtained was added into the column body via a dropper, and thus a concentrating column was obtained.

### Example 3

The column packings of this example were formed from medium-polar macroporous adsorptive resins through static immersion treatment. The medium-polar macroporous adsorptive resins used here were selected from Amberlite XAD-6, Amberlite XAD-7, Amberlite XAD-8 (obtained from Rohm & Hass Company, US). The processing steps were as follows.

1. Immersion, Swelling and Cleansing Treatments

The foregoing medium-polar macroporous adsorptive resins were immersed into ethanol in a container at 20° C. (the amount of ethanol is not strictly limited, so long as it flooded the medium-polar macroporous adsorptive resins completely). An immersion time of 10 hours was enough for swelling. The swelled resins were then rinsed with deionized water, until there was no ethanol in the resins.

2. Static Immersion Treatment with Treating Solution I

Treating solutions were prepared according to the formulations shown in Table 5, using 2-(5-bromo-2-pyridineazo)-5-diethylamino phenol, Na₂B₄O₇.10H₂O, HCl, polyethylene glycol octyl phenyl ether (OP emulsifying agent) and ionized water as raw materials. And then immersed, swelled and purged medium-polar macroporous adsorptive resins were respectively placed into containers containing the foregoing treating solutions, wherein the volume ratio between the medium-polar macroporous adsorptive resins and the treating solution in each container is 1:3. The temperatures and immersion times were shown in Table 5. After immersion treatment, the resins were rinsed with deionized water until the rinsing water became colorless, and as a result column packings according to the present invention were obtained.

TABLE 5

| | Immer- | |
|---|---|---|
| | sion | Immer- |
| | temper- | sion |
| | ature | time |
| Treating Solutions | (° C.) | (min) |
| 0.02 g/L 2-(5-bromo-2-pyridineazo)-5-diethylamino | 20 | 6 |
| phenol, 1.90 g/L Na₂B₄O₇•10H₂O, 6.0 × 10⁻³ mol/L | | |
| HCl, 0.8% polyethylene glycol octyl phenyl ether (OP | | |
| emulsifying agent). | | |
| 0.03 g/L 2-(5-bromo-2-pyridineazo)-5-diethylamino | 20 | 5 |
| phenol, 1.80 g/L Na₂B₄O₇•10H₂O, 6.5 × 10⁻³ mol/L | | |
| HCl, 0.6% polyethylene glycol octyl phenyl ether (OP | | |
| emulsifying agent). | | |
| 0.04 g/L 2-(5-bromo-2-pyridineazo)-5-diethylamino | 20 | 5 |
| phenol, 1.70 g/L Na₂B₄O₇•10H₂O, 7.0 × 10⁻³ mol/L | | |
| HCl, 0.4% polyethylene glycol octyl phenyl ether (OP | | |
| emulsifying agent) | | |
| 0.06 g/L 2-(5-bromo-2-pyridineazo)-5-diethylamino | 20 | 4 |
| phenol, 1.60 g/L Na₂B₄O₇•10H₂O, 8.0 × 10⁻³ mol/L | | |
| HCl, 0.3% polyethylene glycol octyl phenyl ether (OP | | |
| emulsifying agent) | | |

3. Static Immersion Treatment with Treating Solution II

Treating solutions were prepared according to the formulations shown in Table 6, using 2-(5-bromo-2-pyridineazo)-5-diethylamino phenol, glycine, NaOH, NaCl, polyethylene glycol octyl phenyl ether (OP emulsifying agent) and ionized water as raw materials. And then immersed, swelled and purged medium-polar macroporous adsorptive resins were respectively placed into containers containing the foregoing treating solutions, wherein the volume ratio between the medium-polar macroporous adsorptive resins and the treating solution in each container is 1:3. The temperatures and immersion times were shown in Table 6. After immersion treatment, the resins were rinsed with deionized water until the rinsing water became colorless, and as a result column packings according to the present invention were obtained.

TABLE 6

| | Immersion | Immer- |
|---|---|---|
| | temper- | sion |
| | ature | time |
| Treating Solutions | (° C.) | (min) |
| 0.04 g/L 2-(5-bromo-2-pyridineazo)-5-diethylamino | 20 | 6 |
| phenol, 0.08 mol/L glycine, 0.08 mol/L NaOH, | | |
| 0.015 mol/L NaCl, 0.8% polyethylene glycol octyl | | |
| phenyl ether (OP emulsifying agent) | | |
| 0.06 g/L 2-(5-bromo-2-pyridineazo)-5-diethylamino | 20 | 6 |
| phenol, 0.06 mol/L glycine, 0.07 mol/L NaOH, | | |
| 0.012 mol/L NaCl, 0.6% polyethylene glycol octyl | | |
| phenyl ether (OP emulsifying agent) | | |
| 0.08 g/L 2-(5-bromo-2-pyridineazo)-5-diethylamino | 20 | 5 |
| phenol, 0.05 mol/L glycine, 0.06 mol/L NaOH, | | |
| 0.012 mol/L NaCl, 0.5% polyethylene glycol octyl | | |
| phenyl ether (OP emulsifying agent) | | |
| 0.10 g/L 2-(5-bromo-2-pyridineazo)-5-diethylamino | 20 | 5 |
| phenol, 0.05 mol/L glycine, 0.05 mol/L NaOH, | | |
| 0.010 mol/L NaCl, 0.3% polyethylene glycol octyl | | |
| phenyl ether (OP emulsifying agent) | | |

### Example 4

The structure of the concentrating column used in this example was the same with that of Example 2, as shown in FIG. 1, except that column packing 8 was different from that of Example 2. Column packing 8 used here was prepared from medium-polar macroporous adsorptive resins under the trade names of Amberlite XAD-6, Amberlite XAD-7 and Amberlite XAD-8 (obtained from Rohm & Hass company, US) through dynamic on-column treatment or static immersion treatment with treating solution I or treating solution II. Size of the concentrating column is φ5×30 mm.

1. Dynamic On-Column Treatment with Treating Solution I

(1) Preparation of the Mixed Solutions

Treating solutions were prepared according to the formulations shown in Table 7, using 2-(5-bromo-2-pyridineazo)-5-diethylamino phenol, Na₂B₄O₇.10H₂O, HCl, polyethylene glycol octyl phenyl ether (OP emulsifying agent) and ionized water as raw materials.

(2) Packing of the Concentrating Column

After immersion, swelling and cleansing treatments, the medium-polar macroporous adsorbent resins were added into the column body via a dropper, and was washed with deionized water. The swelling and cleaning of the medium-polar macroporous adsorptive resins were conducted in a similar manner as in Example 3.

(3) On-Column Treatment

The treating solutions undiluted or diluted with deionized water according to Table 7 were pumped into the concentrating column at flow rates listed in Table 7 at room temperature (20° C.) until the effluent from concentrating column changed from colorless to light red. And then deionized water was pumped through to wash the concentrating column until the effluent turned from light red to colorless, as a result concentrating columns according to the present invention were obtained.

TABLE 7

| | | Without | | |
|---|---|---|---|---|
| Size of the | | dilution or | Flow rate on | Time on |
| concentrating | | folds of | column | column |
| column | Treating Solutions | dilution | (ml/min) | (min) |
| φ5 × 30 mm | 0.02 g/L 2-(5-bromo-2-pyridineazo)-5-diethylamino | Without | 1.0 | 2 |
| | phenol, 1.90 g/L Na₂B₄O₇•10H₂O, 6.0 × 10⁻³ mol/L HCl, | dilution | | |
| | 0.8% polyethylene glycol octyl phenyl ether (OP | | | |
| | emulsifying agent) | | | |
| φ5 × 30 mm | 0.03 g/L 2-(5-bromo-2-pyridineazo)-5-diethylamino | 2 folds | 1.0 | 3 |
| | phenol, 1.80 g/L Na₂B₄O₇•10H₂O, 6.5 × 10⁻³ mol/L HCl, | | | |
| | 0.6% polyethylene glycol octyl phenyl ether (OP | | | |
| | emulsifying agent) | | | |
| φ5 × 30 mm | 0.04 g/L 2-(5-bromo-2-pyridineazo)-5-diethylamino | 2 folds | 1.0 | 3 |
| | phenol, 1.70 g/L Na₂B₄O₇•10H₂O, 7.0 × 10⁻³ mol/L HCl, | | | |
| | 0.4% polyethylene glycol octyl phenyl ether (OP | | | |
| | emulsifying agent) | | | |
| φ5 × 30 mm | 0.06 g/L 2-(5-bromo-2-pyridineazo)-5-diethylamino | 3 folds | 1.0 | 3 |
| | phenol, 1.60 g/L Na₂B₄O₇•10H₂O, 8.0 × 10⁻³ mol/L HCl, | | | |
| | 0.3% polyethylene glycol octyl phenyl ether (OP | | | |
| | emulsifying agent) | | | |

Note:
The “time on column” in Table 7 refers to the time period during which a treating solution was pumped into the concentrating column.

2. Dynamic On-Column Treatment with Treating Solution II

(1) Preparation of the Mixed Solutions

Treating solutions were prepared according to the formulations shown in Table 8, using 2-(5-bromo-2-pyridineazo)-5-diethylamino phenol, glycine, NaOH, NaCl, polyethylene glycol octyl phenyl ether (OP emulsifying agent) and ionized water as raw materials.

(2) Packing of the Concentrating Column

After immersion, swelling and cleansing treatments, the medium-polar macroporous adsorbent resins were added into the column body via a dropper, and was washed with deionized water. The swelling and cleaning of the medium-polar macroporous adsorptive resins were conducted in a similar manner as in Example 3.

(3) On-Column Treatment

The treating solutions undiluted or diluted with deionized water according to Table 8 were pumped into the concentrating column at flow rates listed in Table 8 at room temperature (20° C.) until the effluent from concentrating column changed from colorless to light red. And then deionized water was pumped through to wash the concentrating column until the effluent turned from light red to colorless, as a result concentrating columns according to the present invention were obtained.

TABLE 8

| | | Without | | |
|---|---|---|---|---|
| Size of the | | dilution or | Flow rate | Time on |
| concentrating | | folds of | on column | column |
| column | Treating Solutions | dilution | (ml/min) | (min) |
| φ5 × 30 mm | 0.04 g/L 2-(5-bromo-2-pyridineazo)-5-diethylamino | Without | 1.0 | 2 |
| | phenol, 0.08 mol/L glycine, 0.08 mol/L NaOH, | dilution | | |
| | 0.015 mol/L NaCl, 0.8% polyethylene glycol octyl | | | |
| | phenyl ether (OP emulsifying agent) | | | |
| φ5 × 30 mm | 0.06 g/L 2-(5-bromo-2-pyridineazo)-5-diethylamino | Without | 1.0 | 2 |
| | phenol, 0.06 mol/L glycine, 0.07 mol/L NaOH, | dilution | | |
| | 0.012 mol/L NaCl, 0.6% polyethylene glycol octyl | | | |
| | phenyl ether (OP emulsifying agent). | | | |
| φ5 × 30 mm | 0.08 g/L 2-(5-bromo-2-pyridineazo)-5-diethylamino | 2 folds | 1.0 | 2 |
| | phenol, 0.05 mol/L glycine, 0.06 mol/L NaOH, | | | |
| | 0.012 mol/L NaCl, 0.5% polyethylene glycol octyl | | | |
| | phenyl ether (OP emulsifying agent) | | | |
| φ5 × 30 mm | 0.10 g/L 2-(5-bromo-2-pyridineazo)-5-diethylamino | 2 folds | 1.0 | 2 |
| | phenol, 0.05 mol/L glycine, 0.05 mol/L NaOH, | | | |
| | 0.010 mol/L NaCl, 0.3% polyethylene glycol octyl | | | |
| | phenyl ether (OP emulsifying agent) | | | |

Note:
The “time on column” in Table 8 refers to the time period during which a treating solution was pumped into the concentrating column.

3. Static Immersion Treatment with Treating Solution I

After immersion, swelling and cleansing treatments, the medium-polar macroporous adsorbent resins were subjected to static immersion treatment with treating solution I in a similar manner as in Example 3. The column packing thus obtained was added into the column body via a dropper, and thus a concentrating column was obtained.

4. Static Immersion Treatment with Treating Solution II

After immersion, swelling and cleansing treatments, the medium-polar macroporous adsorbent resins were subjected to static immersion treatment with treating solution II in a similar manner as in Example 3. The column packing thus obtained was added into the column body via a dropper, and thus a concentrating column was obtained.

### Example 5

The column packings of this example were formed from polar macroporous adsorptive resin through static immersion. The polar macroporous adsorptive resins used here were selected from Amberlite XAD-9 and Amberlite XAD-10 (obtained from Rohm & Hass company, US) and NKA (obtained from Nankai University, Tianjin, China). The processing steps were as follows:

1. Immersion, Swelling and Cleansing Treatments

The foregoing polar macroporous adsorptive resins were immersed into ethanol in a container at 30° C. (the amount of ethanol is not strictly limited, so long as it flooded the medium-polar macroporous adsorptive resins completely). An immersion time of 8 hours was enough for swelling. The swelled resins were then rinsed with deionized water, until there was no ethanol in the resins.

2. Static Immersion Treatment with Treating Solution I

Treating solutions were prepared according to the formulations shown in Table 9, using 2-(5-bromo-2-pyridineazo)-5-diethylamino phenol, Na₂B₄O₇10H₂O, HCl, polyethylene glycol octyl phenyl ether (OP emulsifying agent) and ionized water as raw materials. And then immersed, swelled and purged polar macroporous adsorptive resins were respectively placed into containers containing the foregoing treating solutions, wherein the volume ratio between the polar macroporous adsorptive resins and the treating solution in each container is 1:2. The temperatures and immersion times were shown in Table 9. After immersion treatment, the resins were rinsed with deionized water until the rinsing water became colorless, and as a result column packings according to the present invention were obtained.

TABLE 9

| | Immer- | |
|---|---|---|
| | sion | Immer- |
| | temper- | sion |
| | ature | time |
| Treating Solutions | (° C.) | (min) |
| 0.02 g/L 2-(5-bromo-2-pyridineazo)-5-diethylamino | 30 | 5 |
| phenol, 1.90 g/L Na₂B₄O₇•10H₂O, 6.0 × 10⁻³ mol/L | | |
| HCl, 0.8% polyethylene glycol octyl phenyl ether (OP | | |
| emulsifying agent) | | |
| 0.03 g/L 2-(5-bromo-2-pyridineazo)-5-diethylamino | 30 | 4 |
| phenol, 1.80 g/L Na₂B₄O₇•10H₂O, 6.5 × 10⁻³ mol/L | | |
| HCl, 0.6% polyethylene glycol octyl phenyl ether (OP | | |
| emulsifying agent) | | |
| 0.04 g/L 2-(5-bromo-2-pyridineazo)-5-diethylamino | 30 | 4 |
| phenol, 1.70 g/L Na₂B₄O₇•10H₂O, 7.0 × 10⁻³ mol/L | | |
| HCl, 0.4% polyethylene glycol octyl phenyl ether (OP | | |
| emulsifying agent) | | |
| 0.06 g/L 2-(5-bromo-2-pyridineazo)-5-diethylamino | 30 | 4 |
| phenol, 1.60 g/L Na₂B₄O₇•10H₂O, 8.0 × 10⁻³ mol/L | | |
| HCl, 0.3% polyethylene glycol octyl phenyl ether (OP | | |
| emulsifying agent) | | |

3. Static Immersion Treatment with Treating Solution II

Treating solutions were prepared according to the formulations shown in Table 10, using 2-(5-bromo-2-pyridineazo)-5-diethylamino phenol, glycine, NaOH, NaCl, polyethylene glycol octyl phenyl ether (OP emulsifying agent) and ionized water as raw materials. And then immersed, swelled and purged polar macroporous adsorptive resins were respectively placed into containers containing the foregoing treating solutions, wherein the volume ratio between the polar macroporous adsorptive resins and the treating solution in each container is 1:2. The temperatures and immersion times were shown in Table 10. After immersion treatment, the resins were rinsed with deionized water until the rinsing water became colorless, and as a result column packings according to the present invention were obtained.

TABLE 10

| | Immersion | Immer- |
|---|---|---|
| | temper- | sion |
| | ature | time |
| Treating Solutions | (° C.) | (min) |
| 0.04 g/L 2-(5-bromo-2-pyridineazo)-5-diethylamino | 30 | 4 |
| phenol, 0.08 mol/L glycine, 0.08 mol/L NaOH, | | |
| 0.015 mol/L NaCl, 0.8% polyethylene glycol octyl | | |
| phenyl ether (OP emulsifying agent) | | |
| 0.06 g/L 2-(5-bromo-2-pyridineazo)-5-diethylamino | 30 | 4 |
| phenol, 0.06 mol/L glycine, 0.07 mol/L NaOH, | | |
| 0.012 mol/L NaCl, 0.6% polyethylene glycol octyl | | |
| phenyl ether (OP emulsifying agent) | | |
| 0.08 g/L 2-(5-bromo-2-pyridineazo)-5-diethylamino | 30 | 4 |
| phenol, 0.05 mol/L glycine, 0.06 mol/L NaOH, | | |
| 0.012 mol/L NaCl, 0.5% polyethylene glycol octyl | | |
| phenyl ether (OP emulsifying agent) | | |
| 0.10 g/L 2-(5-bromo-2-pyridineazo)-5-diethylamino | 30 | 4 |
| phenol, 0.05 mol/L glycine, 0.05 mol/L NaOH, | | |
| 0.010 mol/L NaCl, 0.3% polyethylene glycol octyl | | |
| phenyl ether (OP emulsifying agent) | | |

### Example 6

The structure of the concentrating column used in this example is the same with that of Example 2, as shown in FIG. 1, except that the column packing 8 used here was different from that of Example 2. Column packing 8 used in this example was prepared from polar macroporous adsorptive resin under the trade names of Amberlite XAD-9 and Amberlite XAD-10 (obtained from Rohm & Hass company, US) and NKA (produced by Nankai University, Tianjin, China) through dynamic on-column treatment or static immersion treatment with treating solution I or treating solution II. Size of the concentrating column is φ5×30 mm.

1. Dynamic on-Column Treatment with Treating Solution I

(1) Preparation of the Mixed Solutions

Treating solutions were prepared according to the formulations shown in Table 11, using 2-(5-bromo-2-pyridineazo)-5-diethylamino phenol, Na₂B₄O₇.10H₂O, HCl, polyethylene glycol octyl phenyl ether (OP emulsifying agent) and ionized water as raw materials

(2) Packing of the Concentrating Column

After immersion, swelling and cleansing treatments, the polar macroporous adsorbent resins were added into the column body via a dropper, and washed with deionized water. The swelling and cleaning of the polar macroporous adsorptive resins were conducted in a similar manner as in Example 5.

(3) On-Column Treatment

The treating solutions undiluted or diluted with deionized water according to Table 11 were pumped into the concentrating column at flow rates listed in Table 11 at room temperature (30° C.) until the effluent from concentrating column changed from colorless to light red. And then deionized water was pumped through to wash the concentrating column until the effluent turned from light red to colorless, as a result concentrating columns according to the present invention were obtained.

TABLE 11

| Size of the | | Without dilution | Flow rate on | Time on |
|---|---|---|---|---|
| concentrating | | or | column | column |
| column | Treating Solutions | folds of dilution | (ml/min) | (min) |
| φ5 × 30 mm | 0.02 g/L 2-(5-bromo-2-pyridineazo)-5-diethylamino phenol, | Without | 1.0 | 2 |
| | 1.90 g/L Na₂B₄O₇•10H₂O, 6.0 × 10⁻³ mol/L HCl, | dilution | | |
| | 0.8% polyethylene glycol octyl phenyl ether (OP emulsifying | | | |
| | agent) | | | |
| φ5 × 30 mm | 0.03 g/L 2-(5-bromo-2-pyridineazo)-5-diethylamino phenol, | 2 folds | 1.0 | 3 |
| | 1.80 g/L Na₂B₄O₇•10H₂O, 6.5 × 10⁻³ mol/L HCl, | | | |
| | 0.6% polyethylene glycol octyl phenyl ether (OP emulsifying | | | |
| | agent) | | | |
| φ5 × 30 mm | 0.04 g/L 2-(5-bromo-2-pyridineazo)-5-diethylamino phenol, | 2 folds | 1.0 | 3 |
| | 1.70 g/L Na₂B₄O₇•10H₂O, 7.0 × 10⁻³ mol/L HCl, | | | |
| | 0.4% polyethylene glycol octyl phenyl ether (OP emulsifying | | | |
| | agent) | | | |
| φ5 × 30 mm | 0.06 g/L 2-(5-bromo-2-pyridineazo)-5-diethylamino phenol, | 3 folds | 1.0 | 3 |
| | 1.60 g/L Na₂B₄O₇•10H₂O, 8.0 × 10⁻³ mol/L HCl, | | | |
| | 0.3% polyethylene glycol octyl phenyl ether (OP emulsifying | | | |
| | agent) | | | |

Note:
The “time on column” in Table 11 refers to the time period during which a treating solution was pumped into the concentrating column.

2. Dynamic On-Column Treatment with Treating Solution II

(1) Preparation of the Mixed Solutions

Treating solutions were prepared according to the formulations shown in Table 12, using 2-(5-bromo-2-pyridineazo)-5-diethylamino phenol, glycine, NaOH, NaCl, polyethylene glycol octyl phenyl ether (OP emulsifying agent) and ionized water as raw materials.

(2) Packing of the Concentrating Column

After immersion, swelling and cleansing treatments, the polar macroporous adsorbent resins were added into the column body via a dropper, and washed with deionized water. The swelling and cleaning of the polar macroporous adsorptive resins were conducted in a similar manner as in Example 5.

(3) On-Column Treatment

The treating solutions undiluted or diluted with deionized water according to Table 12 were pumped into the concentrating column at flow rates listed in Table 12 at room temperature (30° C.) until the effluent from concentrating column changed from colorless to light red. And then deionized water was pumped through to wash the concentrating column until the effluent turned from light red to colorless, as a result concentrating columns according to the present invention were obtained.

TABLE 12

| Size of the | | Without dilution | Flow rate | Time on |
|---|---|---|---|---|
| concentrating | | or | on column | column |
| column | Treating Solutions | folds of dilution | (ml/min) | (min) |
| φ5 × 30 mm | 0.04 g/L 2-(5-bromo-2-pyridineazo)-5-diethylamino phenol, | Without | 1.0 | 2 |
| | 0.08 mol/L glycine, 0.08 mol/L NaOH, 0.015 mol/L NaCL, | dilution | | |
| | 0.8% polyethylene glycol octyl phenyl ether (OP emulsifying | | | |
| | agent) | | | |
| φ5 × 30 mm | 0.06 g/L 2-(5-bromo-2-pyridineazo)-5-diethylamino phenol, | Without dilution | 1.0 | 2 |
| | 0.06 mol/L glycine, 0.07 mol/L NaOH, 0.012 mol/L NaCl, | | | |
| | 0.6% polyethylene glycol octyl phenyl ether (OP emulsifying | | | |
| | agent) | | | |
| φ5 × 30 mm | 0.08 g/L 2-(5-bromo-2-pyridineazo)-5-diethylamino phenol, | 2 folds | 1.0 | 2 |
| | 0.05 mol/L glycine, 0.6 mol/L NaOH, 0.012 mol/L NaCl, | | | |
| | 0.5% polyethylene glycol octyl phenyl ether (OP emulsifying | | | |
| | agent) | | | |
| φ5 × 30 mm | 0.10 g/L 2-(5-bromo-2-pyridineazo)-5-diethylamino phenol, | 2 folds | 1.0 | 2 |
| | 0.05 mol/L glycine, 0.05 mol/L NaOH, 0.010 mol/L NaCl, | | | |
| | 0.3% polyethylene glycol octyl phenyl ether (OP emulsifying | | | |
| | agent) | | | |

Note:
The “time on column” in Table 12 refers to the time period during which a treating solution was pumped into the concentrating column.

3. Static Immersion Treatment with Treating Solution I

After immersion, swelling and cleansing treatments, the polar macroporous adsorbent resins were subjected to static immersion treatment with treating solution I in a similar manner as in Example 5. The column packing thus obtained was added into the column body via a dropper, and thus a concentrating column was obtained

4. Static Immersion Treatment with Treating Solution II

After immersion, swelling and cleansing treatments, the polar macroporous adsorbent resins were subjected to static immersion treatment with treating solution II in a similar manner as in Example 5. The column packing thus obtained was added into the column body via a dropper, and thus a concentrating column was obtained.

### Example 7

In this example, column packings and concentrating columns according to the present invention were compared with those disclosed in China patent ZL 200410040361.5.

1. Static Immersion Treatments of Non-Polar, Medium-Polar and Polar Macroporous Adsorptive Resins

TABLE 13
Treating solutions of China patent ZL 200410040361.5

| | | Volume ratio between | Immersion | Immersion |
|---|---|---|---|---|
| | | the resin and treating | temperature | time |
| Types of macroporous adsorptive resin | Type of the treating solutions | solution | (° C.) | (min) |
| 1. non-polar (Amberlite XAD-1) | 3 mol/L NH₄OH-1 mol/L HAC- | 1:40 | 25 | 40 |
| | 4.0 × 10⁻⁴ mol/L pyridine-(2-azo-4-)resorcinol | | | |
| 2. medium-polar (Amberlite XAD-6) | 3 mol/L NH₄OH-1 mol/L HAC- | 1:40 | 25 | 40 |
| | 4.0 × 10⁻⁴ mol/L pyridine-(2-azo-4-) resorcinol | | | |
| 3. polar (Amberlite XAD-9) | 3 mol/L NH₄OH-1 mol/L HAC- | 1:40 | 25 | 40 |
| | 4.0 × 10⁻⁴ mol/L pyrindine-(2-azo-4-)resorcinol | | | |

TABLE 14
Treating solutions according to the present invention

| | | Volume ratio | | |
|---|---|---|---|---|
| | | between the resin | Immersion | Immersion |
| | | and treating | temperature | time |
| Types of macroporous adsorptive resin | Type of the treating solutions | solution | (° C.) | (min) |
| 1. non-polar (Amberlite XAD-1) | 0.04 g/L 2-(5-bromo-2-pyridineazo)-5-diethylamino phenol, | 1:4 | 25 | 4 |
| | 1.70 g/L Na₂B₄O₇•10H₂O, 7.0 × 10⁻³ mol/L HCl, | | | |
| | 0.4% polyethylene glycol octyl phenyl ether (OP emulsifying | | | |
| | agent) | | | |
| 2. medium-polar(Amberlite XAD-6) | 0.04 g/L 2-(5-bromo-2-pyridineazo)-5-diethylamino phenol, | 1:4 | 25 | 4 |
| | 1.70 g/L Na₂B₄O₇•10H₂O, 7.0 × 10⁻³ mol/L HCl, | | | |
| | 0.4% polyethylene glycol octyl phenyl ether (OP emulsifying | | | |
| | agent) | | | |
| 3. polar (Amberlite XAD-9) | 0.04 g/L 2-(5-bromo-2-pyridineazo)-5-diethylamino phenol, | 1:4 | 25 | 4 |
| | 1.70 g/L Na₂B₄O₇•10H₂O, 7.0 × 10⁻³ mol/L HCl, | | | |
| | 0.4% polyethylene glycol octyl phenyl ether (OP emulsifying | | | |
| | agent) | | | |
| 4. non-polar (Amberlite XAD-1) | 0.06 g/L 2-(5-bromo-2-pyridineazo)-5-diethylamino phenol, | 1:2 | 25 | 4 |
| | 0.06 mol/L glycine, 0.07 mol/L NaOH, | | | |
| | 0.012 mol/L NaCl, 0.6% polyethylene glycol octyl phenyl ether | | | |
| | (OP emulsifying agent). | | | |
| 5. medium-polar (Amberlite XAD-6) | 0.06 g/L 2-(5-bromo-2-pyridineazo)-5-diethylamino phenol, | 1:2 | 25 | 4 |
| | 0.06 mol/L glycine, 0.07 mol/L NaOH, | | | |
| | 0.012 mol/L NaCl, 0.6% polyethylene glycol octyl phenyl ether | | | |
| | (OP emulsifying agent). | | | |
| 6. polar (Amberlite XAD-9) | 0.06 g/L 2-(5-bromo-2-pyridineazo)-5-diethylamino phenol, | 1:2 | 25 | 4 |
| | 0.06 mol/L glycine, 0.07 mol/L NaOH, | | | |
| | 0.012 mol/L NaCl, 0.6% polyethylene glycol octyl phenyl ether | | | |
| | (OP emulsifying agent) | | | |

2. Dynamic On-Column Treatments of Non-Polar, Medium-Polar and Polar Macroporous Adsorptive Resins

TABLE 15
Treating solutions of China patent ZL 200410040361.5

| | | Size of the | Flow rate on | Time on |
|---|---|---|---|---|
| | | concentrating | column | column |
| Types of macroporous adsorptive resin | Types of the treating solutions | column | (ml/min) | (min) |
| 1. non-polar (Amberlite XAD-1) | 3 mol/L NH₄OH-1 mol/L HAC- | φ5 × 30 mm | 1.0 | 40 |
| | 4.0 × 10⁻⁴ mol/L pyridine-(2-azo-4-)resorcinol | | | |
| 2. medium-polar (Amberlite XAD-6) | 3 mol/L NH₄OH-1 mol/L HAC- | φ5 × 30 mm | 1.0 | 40 |
| | 4.0 × 10⁻⁴ mol/L pyridine-(2-azo-4-)resorcinol | | | |
| 3. polar (Amberlite XAD-9) | 3 mol/L NH₄OH-1 mol/L HAC- | φ5 × 30 mm | 1.0 | 40 |
| | 4.0 × 10⁻⁴ mol/L pyridine-(2-azo-4-)resorcinol | | | |

Note:
The “time on column” in Table 15 refers to the time period during which a treating solution was pumped into the concentrating column.

TABLE 16
Treating solutions according to the present invention

| | | Size of the | Flow rate on | Time on |
|---|---|---|---|---|
| | | concentrating | column | column |
| Types of macroporous adsorptive resin | Types of the treating solutions | column | (ml/min) | (min) |
| 1. non-polar(Amberlite XAD-1) | 0.04 g/L 2-(5-bromo-2-pyridineazo)-5-diethylamino phenol, | φ5 × 30 mm | 1.0 | 2 |
| | 1.70 g/L Na₂B₄O₇•10H₂O, 9.0 × 10⁻³ mol/L HCl, 0.4% polyethylene | | | |
| | glycol octyl phenyl ether (OP emulsifying agent) | | | |
| 2. medium-polar (Amberlite XAD-6) | 0.04 g/L 2-(5-bromo-2-pyridineazo)-5-diethylamino phenol, | φ5 × 30 mm | 1.0 | 2 |
| | 1.70 g/L Na₂B₄O₇•10H₂O, 9.0 × 10⁻³ mol/L HCl, 0.4% polyethylene | | | |
| | glycol octyl phenyl ether (OP emulsifying agent) | | | |
| 3. polar (Amberlite XAD-9) | 0.04 g/L 2-(5-bromo-2-pyridineazo)-5-diethylamino phenol, | φ5 × 30 mm | 1.0 | 2 |
| | 1.70 g/L Na₂B₄O₇•10H₂O, 9.0 × 10⁻³ mol/L HCl, 0.4% polyethylene | | | |
| | glycol octyl phenyl ether (OP emulsifying agent) | | | |
| 4. non-polar(Amberlite XAD-1) | 0.06 g/L 2-(5-bromo-2-pyridineazo)-5-diethylamino phenol, | φ5 × 30 mm | 1.0 | 2 |
| | 0.06 mol/L glycine, 0.07 mol/L NaOH, 0.012 mol/L NaCl, | | | |
| | 0.6% polyethylene glycol octyl phenyl ether (OP emulsifying | | | |
| | agent) | | | |
| 5. medium-polar (Amberlite XAD-6) | 0.06 g/L 2-(5-bromo-2-pyridineazo)-5-diethylamino phenol, | φ5 × 30 mm | 1.0 | 2 |
| | 0.06 mol/L glycine, 0.07 mol/L NaOH, 0.012 mol/L NaCl, | | | |
| | 0.6% polyethylene glycol octyl phenyl ether (OP emulsifying | | | |
| | agent) | | | |
| 6. polar (Amberlite XAD-9) | 0.06 g/L 2-(5-bromo-2-pyridineazo)-5-diethylamino phenol, | φ5 × 30 mm | 1.0 | 2 |
| | 0.06 mol/L glycine, 0.07 mol/L NaOH, 0.012 mol/L NaCl, | | | |
| | 0.6% polyethylene glycol octyl phenyt ether (OP emulsifying | | | |
| | agent) | | | |

Note:
The “time on column” in Table 16 refers to the time period during which a treating solution was pumped into the concentrating column.

The experimental data in Tables 13-16 indicate that, compared with China patent ZL 200410040361.5, the present invention needs less time, less consumption of treating solutions in the static immersion treatment or dynamic on-column treatment of the macroporous adsorptive resins, and the cost of the column packings and concentrating columns were in turn reduced.

1. Column packing useful for the analysis of metallic elements, characterized in that the column packing is formed from swollen and cleaned macroporous adsorbent resins of high, medium or non-polarity via static immersion treatment using treating solution I or treating solution II;
said treating solution I is a mixed aqueous solution of 0.02˜0.06 g/L of 2-(5-bromo-2-pyridyl azo)-5-diethylamino phenol, 1.60˜1.90 g/L of Na₂B₄O₇.10H₂O, 6.0×10⁻³˜8.0×10⁻³ mol/L of HCl, and polyethylene glycol octyl phenyl ether with a volume percent of 0.3˜0.8%;
said treating solution II is a mixed aqueous solution of 0.04˜0.10 g/L of 2-(5-bromo-2-pyridyl azo)-5-diethylamino phenol, 0.05˜0.08 mol/L of glycin, 0.05˜0.08 mol/L of NaOH, 0.010˜0.015 mol/L of NaCl, and polyethylene glycol octyl phenyl ether with a volume percent of 0.3˜0.8%.

2. The column packing useful for the analysis of metallic elements according to claim 1, wherein the static immersion treatment comprises placing the swollen and cleaned macroporous adsorbent resins of high, medium or non-polarity into treating solution I or treating solution II, immersing at room temperature for at least 4 minutes, and then rinsing it with deionized water till the rinsing water becomes colorless.

3. The column packing useful for the analysis of metallic elements according to claim 1, wherein the volume ratio between the swollen and cleaned macroporous adsorbent resins of high, medium or non-polarity and the treating solution I or treating solution II is 1:2˜1:4.

4. A concentrating column for the analysis of metallic elements, essentially consisting of a column body, filtration membranes (6) installed at the inner sides of the inlet and outlet of the column body, and column packing (8) packed inside the column body, characterized in that the column packing (8) is formed from swollen and cleaned macroporous adsorbent resins of high, medium or non-polarity via dynamic on-column treatment or static immersion treatment using treating solution I or treating solution II;
said treating solution I is a mixed aqueous solution of 0.02˜0.06 g/L of 2-(5-bromo-2-pyridyl azo)-5-diethylamino phenol, 1.60˜1.90 g/L of Na₂B₄O₇.10H₂O, 6.0×10⁻³˜8.0×10⁻³ mol/L of HCl, and polyethylene glycol octyl phenyl ether with a volume percent of 0.3˜0.8%;
said treating solution II is a mixed aqueous solution of 0.04˜0.10 g/L of 2-(5-bromo-2-pyridyl azo)-5-diethylamino phenol, 0.05˜0.08 mol/L of glycin, 0.05˜0.08 mol/L of NaOH, 0.010˜0.015 mol/L of NaCl, and polyethylene glycol octyl phenyl ether with a volume percent of 0.3˜0.8%.

5. The concentrating column for the analysis of metallic elements according to claim 4, wherein said dynamic on-column treatment comprises packing the swollen and cleaned macroporous adsorbent resins of high, medium or non-polarity into the column, then pumping treating solution I or treating solution II into the concentrating column directly or after 2˜4 folds dilution using deionized water, until the effluent from the concentrating column changes from colorless to light red, then pumping deionized water into the concentrating column, until the effluent from the concentrating column changes from light red to colorless.

6. The concentrating column for the analysis of metallic elements according to claim 4, wherein said static immersion treatment comprises placing the swollen and cleaned macroporous adsorbent resins of high, medium or non-polarity into treating solution I or treating solution II, immersing them at room temperature for at least 4 minutes, and then rinsing them with deionized water till the rinsing water becomes colorless.

7. The concentrating column for the analysis of metallic elements according to claim 4, wherein during the static immersion treatment, the volume ratio between the swollen and cleaned macroporous adsorbent resins of high, medium or non-polarity and the treating solution I or treating solution II is 1:2˜1:4.

8. The column packing useful for the analysis of metallic elements according to claim 2, wherein the volume ratio between the swollen and cleaned macroporous adsorbent resins of high, medium or non-polarity and the treating solution I or treating solution II is 1:2˜1:4.

9. The concentrating column for the analysis of metallic elements according to claim 6, wherein during the static immersion treatment, the volume ratio between the swollen and cleaned macroporous adsorbent resins of high, medium or non-polarity and the treating solution I or treating solution II is 1:2˜1:4.
